(19) United States
(12) Patent Application Publication
(10) Pub. No.: US 2011/0108038 A1
(43) Pub. Date: May 12, 2011

(54) ENDOTRACHEAL TUBE SECURING DEVICE

(76) Inventor: Marion Pierson, Lee's Summit, MO (US)

(21) Appl. No.: 12/841,826

(22) Filed: Jul. 22, 2010

Related U.S. Application Data
(60) Provisional application No. 61/259,317, filed on Nov. 9, 2009.

(51) Int. Cl. A61M 16/04

(52) U.S. Cl. 128/207.14

(57) ABSTRACT

An endotracheal tube securing device having a securing device body that is operatively engaged to a biased plunger for securing an endotracheal tube to the securing device body is disclosed. The securing device body defines a chamber in communication with opposing front and rear openings with a top opening in transverse relation to the front and rear openings. A plunger biased by a spring is slidably disposed inside the chamber through a top opening and includes a channel that communicates with first and second opposed openings that become aligned with the front and rear openings of the securing device body when the user depresses the plunger and inserts the endotracheal tube through the aligned plunger. The endotracheal tube becomes secured to the securing device body when the user releases the biased plunger, thereby engaging the endotracheal tube between the plunger and the securing device body.

### CROSS REFERENCE TO RELATED APPLICATIONS

This application claims priority from U.S. provisional patent application Ser. No. 61/259,317 filed on Nov. 9, 2009.

### FIELD

This application relates to securing devices, and in particular a securing device for securing medical tubing to a patient during an intubation procedure.

### BACKGROUND

Tracheal intubation is the placement of a flexible plastic tube into the trachea to protect the patient's airway and provide a means of mechanical ventilation when the tube is connected to a ventilator apparatus. The most common tracheal intubation procedure is orotracheal intubation where an endotracheal tube is passed through the mouth, larynx, and vocal cords, and into the trachea. Tracheal intubation can be a potentially dangerous invasive procedure since it requires that the endotracheal tube be properly inserted into the trachea, especially in pediatric patients because of the significant anatomical and physiological differences between children and adults. In a pediatric intubation procedure, it is critical that the tip of the endotracheal tube be at midtrachea (between the clavicles on an AP chest X-ray) with the position of the tube being checked by auscultation (e.g., equal air entry on each side and, in long-term intubation, by chest X-ray). Once the endotracheal tube is inserted into the trachea, it is critical that the tube be properly secured to the pediatric patient in order to prevent removal or further insertion of the endotracheal tube. Although, prior art endotracheal tube securing devices have been used to secure endotracheal tubes to patients using various systems and methods, there is still a need in the art for further advances and improvements in an endotracheal tube securing devices.

### SUMMARY

In an embodiment, a securing device may include a securing device body defining a chamber in communication with a front opening and an opposing rear opening with the front and rear openings being in transverse relationship with a top opening. A plunger is slidably disposed inside the chamber through the top opening and defines a channel in communication with first and second opposed openings. A elongate tube may be inserted through the channel of the plunger when the first and second opposed openings of the plunger are aligned with the front and rear openings of the securing device body. In addition, a spring is disposed inside the chamber and engaged to the plunger for biasing the plunger when the plunger is depressed such that the front and rear opposed openings of the securing device body are aligned with the first and second opposed openings of the plunger for permitting insertion of the elongate tube through the plunger.

In another embodiment, an endotracheal tube securing device may include a securing device body defining a chamber in communication with a front opening and an opposing rear opening with the front and rear openings being in transverse relationship with a top opening. The securing device body further includes a pair of opposed wings for engagement of the endotracheal tube securing device to the patient. A plunger is slidably disposed inside the chamber through the top opening with the plunger defining a channel in communication with first and second opposed openings. An elongate endotracheal tube is inserted through the channel of the plunger when the first and second opposed openings of the plunger are aligned with the front and rear openings of the securing device body. In addition, the endotracheal tube securing device includes a pair of straps with each of the pair of straps having an inner portion adapted to secure a respective wing and an outer portion having an adhesive part for securing the strap to the facial area of a patient. A spring is disposed inside the chamber and engaged to the plunger for biasing the plunger when the plunger is depressed such that the front and rear opposed openings of the securing device body are aligned with the first and second opposed openings of the plunger for permitting insertion of the elongate endotracheal tube through the plunger.

In yet another embodiment, a method of securing an endotracheal tube may include providing a tube securing device comprising a securing device body defining a chamber in communication with a front opening and an opposing rear opening with the front and rear openings being in transverse relationship with a top opening. The tube securing device further includes a plunger that is slidably disposed inside the chamber through the top opening with the plunger defining a channel in communication with first and second opposed openings. An elongate tube is inserted through the channel of the plunger when the first and second opposed openings are aligned with the front and rear openings of the securing device body. In addition, a spring is slidably disposed inside the chamber and engaged to the plunger for biasing the plunger when the plunger is depressed such that the front and rear opposed openings of the securing device body are aligned with the first and second opposed openings of the plunger for permitting insertion of the elongate tube through the plunger. Once the tube securing device is provided, the user depresses the plunger such that the front and rear openings of the securing device body are aligned with the first and second opening of the plunger. The user may insert the elongate tube through the channel of the plunger, and then release the plunger such that the elongate tube is engaged to the tube securing device.

Additional objectives, advantages and novel features will be set forth in the description which follows or will become apparent to those skilled in the art upon examination of the drawings and detailed description which follows.

### BRIEF DESCRIPTION OF THE DRAWINGS

FIG. 1 is an elevated perspective view of an embodiment of the endotracheal tube securing device;

FIG. 2 is a front view of the endotracheal tube securing device;

FIG. 3 is an elevated perspective view of the endotracheal tube securing device showing the plunger and spring arrangement in phantom line;

FIG. 4 is an elevated perspective view of the securing device body;

FIG. 5 is a front view of the securing device body with the chamber shown in phantom line;

FIG. 6 is a top view of the securing device body;

FIG. 7 is an elevated perspective view of the plunger;

FIG. 8 is a front view of the plunger;

FIG. 9 is a top view of the plunger;

FIG. 10 is a front view of the endotracheal tube securing device in the unbiased position;

FIG. 11 is a front view of the endotracheal tube securing device in the biased position;

FIG. 12 is top view of the endotracheal tube securing device in the biased position showing the endotracheal tube inserted through the securing device body;

FIG. 13 is an elevated perspective view of the endotracheal tube securing device in the biased position showing the endotracheal tube inserted through the securing device body;

FIG. 14 is an alternate embodiment of the endotracheal tube securing device;

FIG. 15 is an exploded view of another embodiment of the endotracheal tube securing device illustrating the gripping teeth defined by the plunger;

FIG. 16 is an elevated perspective view of the embodiment of the plunger shown in FIG. 15; and

FIG. 17 is a cross-sectional view of the plunger illustrating the dove-tail protrusion.

Corresponding reference characters indicate corresponding elements among the view of the drawings. The headings used in the figures should not be interpreted to limit the scope of the claims.

### DETAILED DESCRIPTION

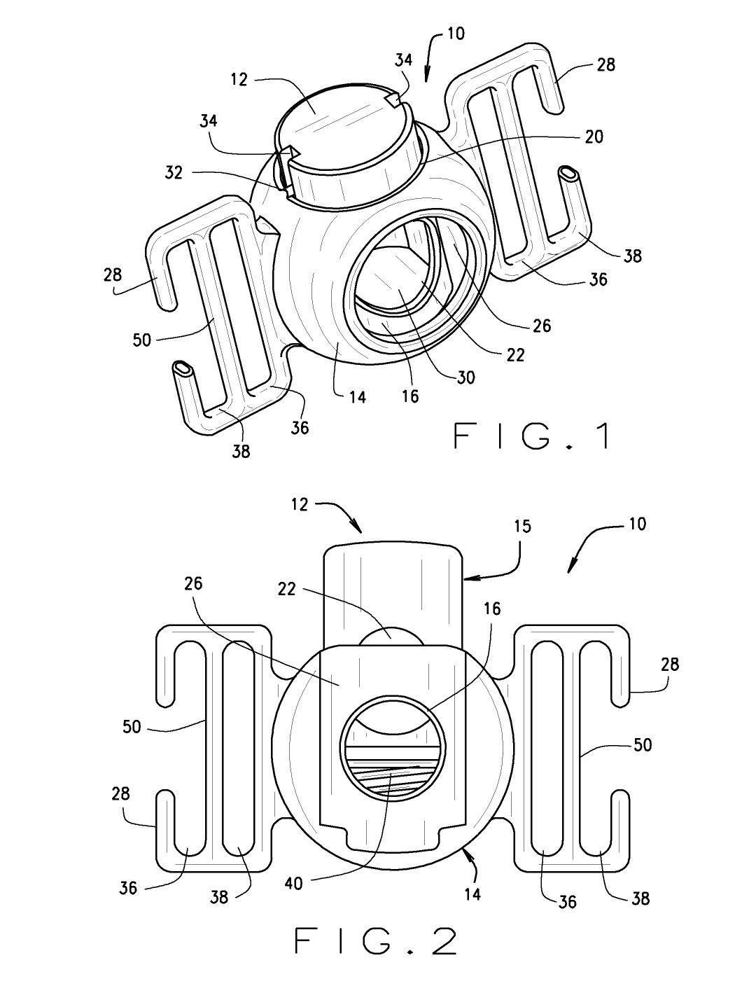

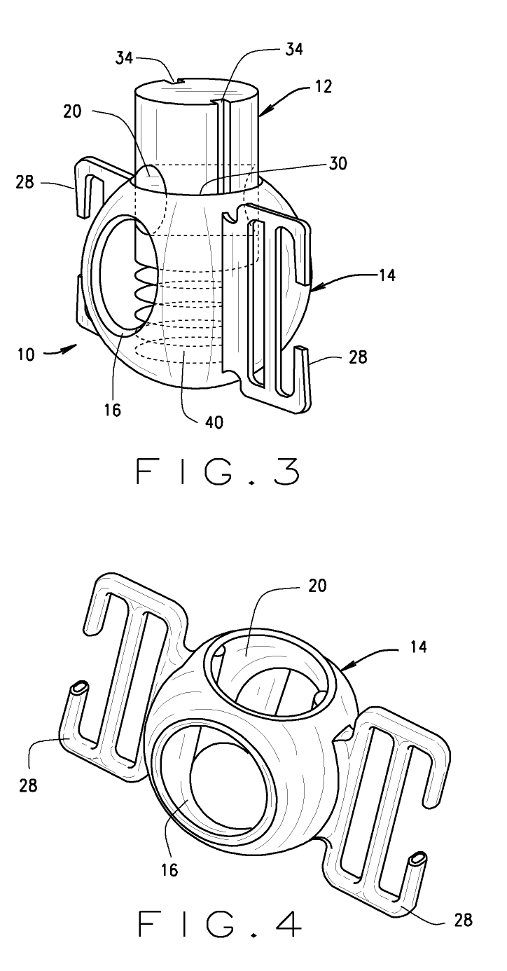

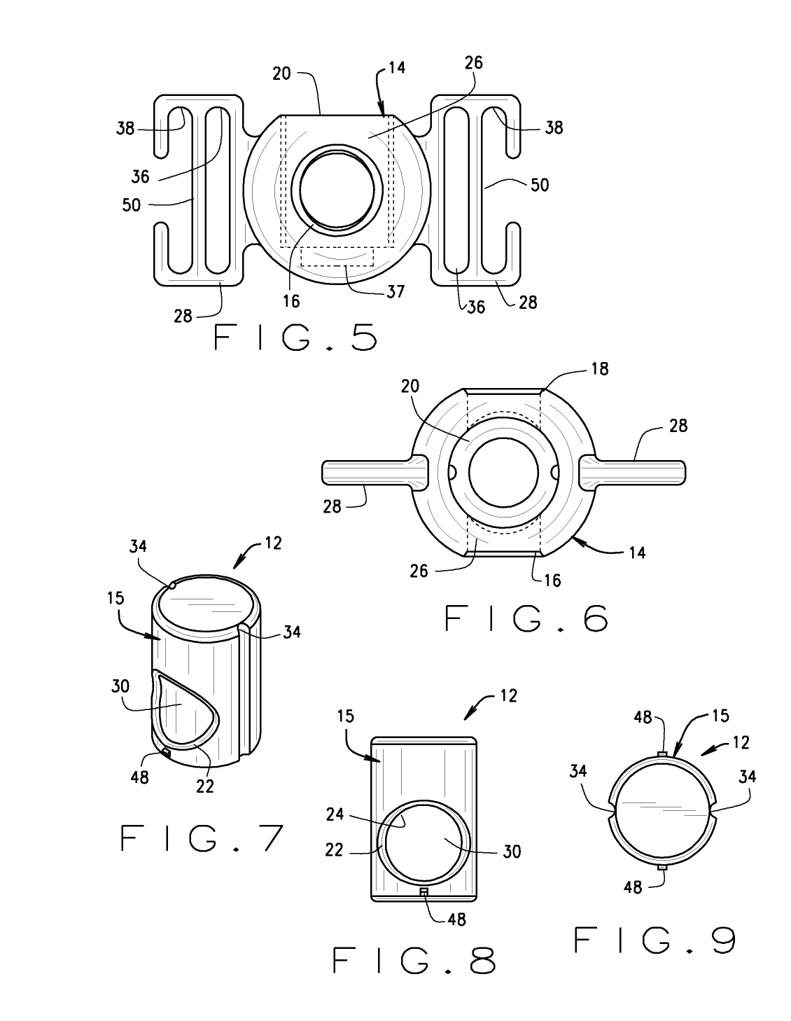

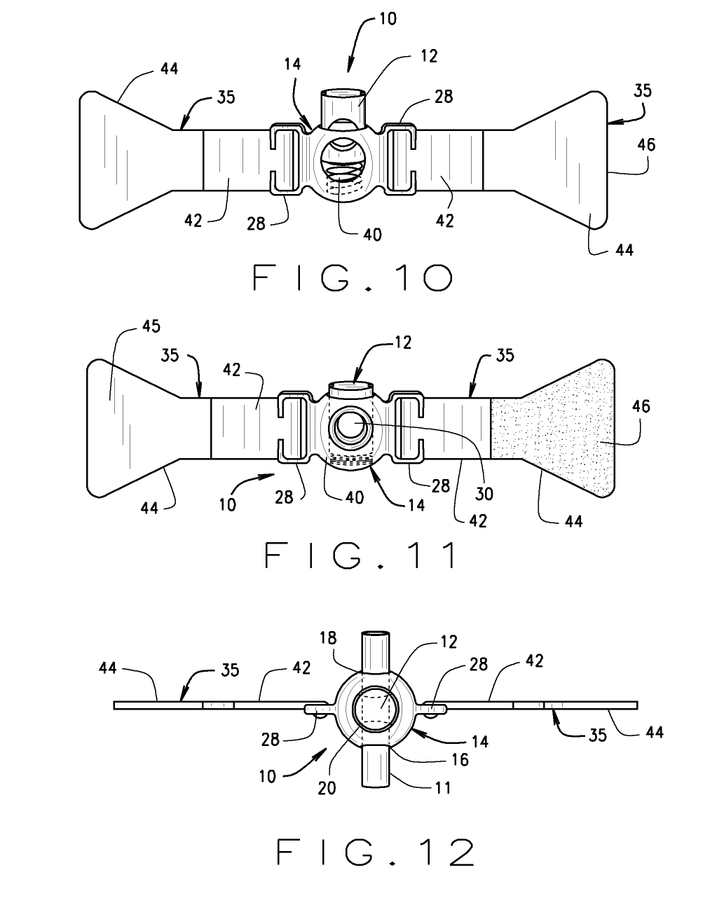

Referring to the drawings, an embodiment of the endotracheal tube securing device for securing an endotracheal tube 11 is illustrated and generally indicated as 10 in FIGS. 1-17. As shown in FIGS. 1, 4, 5 and 6, the endotracheal tube securing device 10 includes a securing device body 14 that defines a chamber 26 in communication with a front opening 16 and an opposing rear opening 18. In addition, the chamber 26 communicates with a top opening 20 that is transverse to the front and rear openings 16 and 18.

The securing device body 14 further includes a pair of opposing wings 28 that extend outwardly from each side of the securing device body 14. Each wing 28 is adapted for attachment to a respective strap 35 for securing the endotracheal tube securing device 10 to the patient's facial area. To secure the straps 35 to the securing device body 14, each strap 35 defines an inner portion 42 having a loop and hook arrangement, such as VELCRO®, for engaging the strap 35 to each respective wing 28. As shown, each wing 28 defines a closed inner loop 36 and an outer semi-open loop 38 for engaging the inner portion 42 of the strap 35 as the loop and hook arrangement part of inner portion 42 is wrapped around the inner loop 36 and outer loop 38 of each wing 28. Referring to FIG. 11, each strap 35 further includes an outer portion 44 having an adhesive flap 46 to attach the outer end of the strap 35 to the facial area of the patient. In one embodiment, the adhesive flap 46 may include a removable cover 45 that is removed prior to attaching the adhesive flap 46 to the patient's facial area.

As shown in FIGS. 1, 7, 8 and 9, a plunger 12 is disposed inside the chamber 26 through the top opening 20 of the securing device body 14. The plunger 12 provides a means for securing the endotracheal tube 11 to the securing device body 14 and includes a plunger body 15 that defines an axial channel 30 that communicates with a first opening 22 at one end of channel 30 and a second opening 24 at the other end of channel 30. The axial channel 30 is sized and shaped to receive the endotracheal tube 11 such that the endotracheal tube 11 may be secured to the endotracheal tube securing device 10 by virtue of the plunger 12 biasing the endotracheal tube 11 against the securing device body 14 as shall be discussed in greater detail below.

Referring specifically to FIGS. 1 and 7, plunger 12 further defines a pair of opposing notches 34 that extends the length of plunger body 15. The notches 34 are adapted to engage respective protrusions 32 defined along opposing sides of the chamber 26 in order to properly orient the plunger 12 inside the securing device body 14 such that the first and second openings 22 and 24 of the plunger 12 are aligned with the front and rear openings 16 and 18 of the securing device body 14. Once so aligned, the endotracheal tube 11 may be inserted through channel 30 of the plunger 12. As shown, the plunger 12 further includes a pair of protrusions 48 along the bottom portion of the plunger that function as stops that prevent the plunger 12 from being completely withdrawn from the securing device body 14 through the top opening 20.

Referring to FIGS. 5, 10, 11 and 12, the chamber 26 of the securing device body 14 communicates with a recess 37 that is adapted to receive a spring 40 for biasing the plunger 12. The plunger 12 is operative between an unbiased position (FIG. 10) and a biased position (FIG. 11). In the unbiased position, the spring 40 is fully extended such that the plunger 12 extends partially through the top opening 20 of the securing device body 14 and the first and second openings 22 and 24 of the plunger 12 are misaligned with the front and rear openings 16 and 18 of the securing device body 14. Conversely, in the biased position, the user forces the plunger 12 downward against the spring 40, thereby compressing the spring 40 such that the plunger 12 is either fully or substantially disposed in the chamber 26 and the first and second openings 22 and 24 of the plunger 12 are aligned with the front and rear openings 16 and 18 of the securing device body 14.

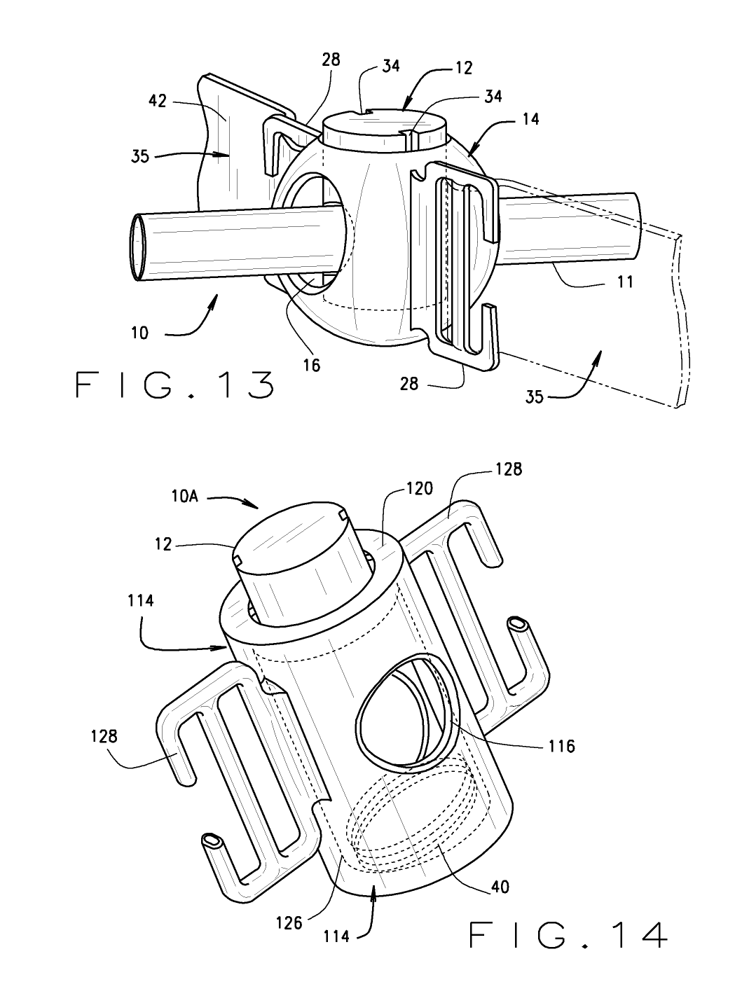

In this biased position, the endotracheal tube 11 may be inserted through the aligned openings 16, 18, 22, and 24 until a predetermined length of the tube 11 extends from the other side of the securing device body 14. In one embodiment, the predetermined length that the endotracheal tube 11 extends from the rear opening 18 of the securing device body 14 matches the length that the endotracheal tube 11 needs to be inserted into the trachea of the patient when the endotracheal tube securing device 10 is attached to the patient's mouth. When the user releases the plunger 12 with the endotracheal tube 11 inserted through the plunger 12, the plunger 12 remains in a biased position as the spring 40 forces the endotracheal tube 11 against the openings 16 and 18 of the securing device body 14 as illustrated in FIGS. 12 and 13. This action secures the endotracheal tube 11 between the plunger 12 and the securing device body 14 until the user presses the plunger 12 slightly downward in order to permit release of the endotracheal tube 11 from the endotracheal tube securing device 10.

Referring to FIGS. 10-13, one method of using the endotracheal tube securing device will be discussed. In FIG. 10, the respective inner portion 42 of each strap 35 is secured to a respective wing 28 by inserting the loop and hook arrangement of the strap 35 through the outer loop 38 and around the inner loop 36 such that the inner portion 42 is wrapped and secured around the arm 50 of the respective wing 28. The securing device body 14 is then mounted on top of the patient's mouth and the removable cover 45 of each adhesive flap 46 of the strap 35 is then removed so that the adhesive flap 46 may be attached to the facial area of the patient when securing the endotracheal tube securing device 10. In one embodiment, each adhesive flap 46 may be applied to a respective cheek area of the patient.

Once the endotracheal tube securing device 10 is secured to the patient, the plunger 12 is depressed by the user to the biased position, thereby aligning the openings 16 and 18 of the securing device body 14 with the openings 22 and 24 of the plunger 12 as shown in FIG. 11. Referring to FIGS. 12 and 13, when the respective openings 16, 18, 22, and 24 are aligned, the user inserts the endotracheal tube 11 through the plunger 12 such that the tube 11 extends outwardly from the rear opening 18 of the securing device body 14 a predetermined distance into the patient's esophagus. The user then releases the plunger 12 such that the spring 40 continues to apply a bias against the plunger 12, which secures the endotracheal tube 11 to the securing body 14. Once the procedure is completed and the endotracheal tube 11 needs to be withdrawn, the user depresses the plunger 12 slightly, which permits the endotracheal tube 11 to be withdrawn through the endotracheal tube securing device 10. In the alternative, the endotracheal tube 11 may be withdrawn by simply disengaging the adhesive flaps 46 and removing the endrotracheal tube securing device 10 from the patient. Although the endotracheal tube 11 may be inserted through the endotracheal tube securing device 10 after the device 10 is secured to the patient, in an alternative embodiment, the endotracheal tube 11 may be inserted through the securing device body 14 prior to attachment of the device 10 to the patient.

Referring to FIG. 14, an alternate embodiment of the endotracheal tube securing device, designated 10A, is illustrated. Endotracheal tube securing device 10A is substantially the same as the other embodiment except the securing device body 114 has a generally cylindrical shape and the opposing wings 128 have a generally rectangular shape. Similarly, the securing device body 114 defines a chamber 126 in communication with opposing front and rear openings 116, 118, while a top opening 120 is defined transverse to openings 116, 118. A plunger 12 is disposed inside the securing device body 114 with a spring 40 disposed inside the chamber 126 that applies a bias against the plunger 12 when the user depresses the plunger 12. A strap (not shown) is also secured to each wing 128 for attaching the endotracheal tube securing device 10A to the patient as described above.

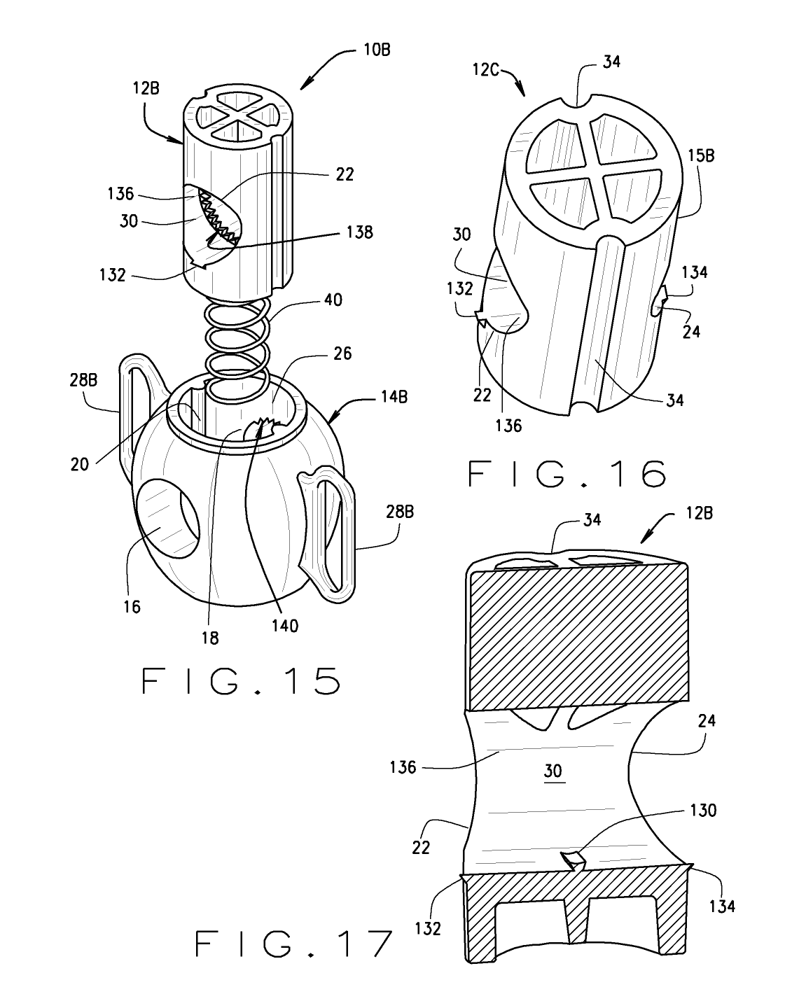

Referring to FIGS. 15 and 16, another embodiment of the securing device, designated 10B, is illustrated. Securing device 10B is substantially similar as the other embodiments except the rear opening 18 of the securing device body 14A may have opposing wings 28B with a closed configuration. In one embodiment, the securing device body 14A may further include a plurality of teeth 140 defined along at least a peripheral edge of rear opening 18. The plurality of teeth 140 is configured to engage and retain the endotracheal tube 11 when the tube 11 is inserted through the plunger 12B when the first and second openings 22 and 24 of plunger 12B are aligned with the front and rear openings 16 and 18 of the securing device body 14B. This arrangement of teeth 140 prevents or inhibits the endotracheal tube 11 from being easily withdrawn from the securing device 10B. In one embodiment, the plunger 12B may also define a plurality of teeth 136 across the axial channel 30 that is also configured to engage and retain the endotracheal tube 11 to the securing device 10B. Referring specifically to FIG. 16, in one embodiment a pair of opposing stops 132 and 134 may be defined adjacent the first opening 22 and second opening 24, respectively, of the plunger 12B. The pair of opposing stops 132 and 134 is configured to prevent the plunger 12B from being completely withdrawn through the opening 20 of the securing device body 14B by engaging the interior surface of the chamber 26 when the plunger 12B has been withdrawn too far from the chamber 26.

Referring to FIG. 17, one embodiment of the plunger 12B may include a dovetail protrusion 130 defined along the channel 30 of the plunger 12B as a means for engaging the endotracheal tube 11 after insertion through the channel 30.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A securing device comprising:
a securing device body defining a chamber in communication with a front opening and an opposing rear opening with the front and rear openings being in transverse relationship with a top opening;
a plunger is slidably disposed inside the chamber through the top opening, the plunger defining a channel in communication with first and second opposed openings;
an elongate tube inserted through the channel of the plunger when the first and second opposed openings of the plunger are aligned with the front and rear openings of the securing device body; and
a spring disposed inside the chamber and engaged to the plunger for biasing the plunger when the plunger is depressed such that the front and rear opposed openings of the securing device body are aligned with the first and second opposed openings of the plunger for permitting insertion of the elongate tube through the plunger.

2. The securing device of claim 1, wherein the plunger defines at least one notch and the securing device body defines at least one protrusion for engagement with the at least one notch for orienting the channel of the plunger relative to the front and back openings of the securing device body.

3. The securing device of claim 1, wherein the plunger defines at least one stop for preventing the removal of the plunger from the chamber of the securing device body.

4. The securing device of claim 1, wherein the securing device body further includes opposing wings, each of the opposing wings defines an inner loop that communicates with an outer loop.

5. The securing device of claim 4, further comprising a pair of straps for securing the securing device body to a patient, each of the pair of straps including an inner portion having a hook and loop arrangement for attachment to the inner and outer loops and an outer portion including an adhesive part for attachment to the facial area of a patient.

6. The securing device of claim 1, wherein the securing device body includes a recess in communication with the chamber with the recess being adapted to receive the spring for biasing the plunger.

7. The securing device of claim 1, wherein the channel of the plunger defines at least one protrusion configured for engaging the elongate tube.

8. The securing device of claim 1, wherein the channel of the plunger defines at least one dovetail protrusion configured for engaging the elongate tube.

9. The securing device of claim 1, wherein the chamber of the securing device body includes a chamber having an inner surface that defines at least one protrusion configured for gripping the elongate tube.

10. The securing device of claim 9, wherein the at least one protrusion is a plurality of teeth configured to engage the elongate tube when inserted through the channel of the plunger.

11. The securing device of claim 1, further comprising:
a plurality of teeth defined around at least one of the front opening and/or rear opening of the securing device body, wherein the plurality of teeth is configured to engage the elongate tube.

12. An endotracheal tube securing device comprising:
a securing device body defining a chamber in communication with a front opening and an opposing rear opening with the front and rear openings being in transverse relationship with a top opening, the securing device body further including a pair of opposed wings;
a plunger is slidably disposed inside the chamber through the top opening, the plunger defining a channel in communication with first and second opposed openings;
an elongate endotracheal tube inserted through the channel of the plunger when the first and second opposed openings of the plunger are aligned with the front and rear openings of the securing device body; and
a pair of straps, each of the pair of straps having an inner portion adapted to secure a respective wing and an outer portion having an adhesive part for securing the strap to the facial area of a patient;
a spring disposed inside the chamber and engaged to the plunger for biasing the plunger when the plunger is depressed such that the front and rear opposed openings of the securing device body are aligned with the first and second opposed openings of the plunger for permitting insertion of the elongate tube through the plunger.

13. A method of using a tube securing device comprising:
providing a tube securing device comprising:
a securing device body defining a chamber in communication with a front opening and an opposing rear opening with the front and rear openings being in transverse relationship with a top opening;
a plunger is slidably disposed inside the chamber through the top opening, the plunger defining a channel in communication with first and second opposed openings;
an elongate tube inserted through the channel of the plunger when the first and second opposed openings are aligned with the front and rear openings of the securing device body; and
a spring slidably disposed inside the chamber and engaged to the plunger for biasing the plunger when the plunger is depressed such that the front and rear opposed openings of the securing device body are aligned with the first and second opposed openings of the plunger for permitting insertion of the elongate tube through the plunger;
depressing the plunger such that the front and rear openings of the securing device body are aligned with the first and second opening of the plunger;
inserting the elongate tube through the channel of the plunger; and
releasing the plunger such that the elongate endotracheal tube is engaged to the tube securing device.

14. The method of claim 11, wherein the spring applies a bias to the plunger when depressing the plunger such that the front and rear openings are aligned with the first and second openings.

15. The method of claim 11, wherein the spring applies a bias to the plunger that engages the elongate endotracheal tube between the plunger and the securing device body.

16. The method of claim 11, further comprising depressing the plunger a second time such that the elongate endotracheal tube is releasable from the plunger.

17. The method of claim 11, wherein depressing the plunger places the plunger in a biased position.
